(19) United States
(12) Patent Application Publication
(10) Pub. No.: US 2024/0192214 A1
(43) Pub. Date: Jun. 13, 2024

(54) COLOR: A LOW-COST OPTODIAGNOSTIC FOR SARS-COV-2

(71) Applicants: THE TRUSTEES OF THE UNIVERSITY OF PENNSYLVANIA, Philadelphia, PA (US); STATE UNIVERSITY OF CAMPINAS (UNICAMP), Campinas (BR)

(72) Inventors: César DE LA FUENTE-NUNEZ, Philadelphia, PA (US); Marcelo DER TOROSSIAN TORRES, Philadelphia, PA (US); Lucas FELIPE DE LIMA, Campinas (BR); André LOPES FERREIRA, Campinas (BR); William REIS DE ARAUJO, Campinas (BR)

(21) Appl. No.: 18/556,385

(22) PCT Filed: Apr. 18, 2022
(86) PCT No.: PCT/US2022/071776

§ 371 Date: Oct. 20, 2023

Related U.S. Application Data
(60) Provisional application No. 63/177,221, filed on Apr. 20, 2021.

(51) Int. Cl. G01N 33/569; G01N 21/78; G01N 33/543

(52) U.S. Cl. CPC G01N 33/56983; G01N 21/78; G01N 33/54346; G01N 2333/165; G01N 2333/948; G01N 2469/10

(57) ABSTRACT

The COVID-19 pandemic has exacerbated society's tremendous health equity gap. Disadvantaged populations have been disproportionally affected by COVID-19, lacking access to affordable testing, a known effective tool for preventing viral spread, hospitalizations, and deaths. Here, we describe COvid-19 Low-cost Optodiagnostic for Rapid testing (COLOR), a colorimetric biosensor fabricated on cotton swabs using gold nanoparticles modified with human angiotensin-converting enzyme 2 (ACE2), which costs 15 ¢ to produce and detects SARS-COV-2 within 5 minutes. COLOR detected very low viral particle loads (limit of detection: 0.154 pg mL⁻¹ of SARS-COV-2 spike protein) and its color intensity correlated with the cycle threshold (Ct) values obtained using RT-PCR. The performance of COLOR was assessed using 100 nasopharyngeal/oropharyngeal (NP/OP) clinical samples, yielding sensitivity, specificity, and accuracy values of 96%, 84%, and 90%, respectively. In summary, each COLOR test can be manufactured for 15 ¢ and presents rapid minute-timescale detection of SARS-COV-2, thus providing a solution to enable high-frequency testing, particularly in low-resource communities.

### RELATED APPLICATIONS

The present application claims priority to and the benefit of U.S. patent application No. 63/177,221, “COLOR: A Low-Cost Optodiagnostic For SARS-CoV-2” (filed Apr. 20, 2021), the entirety of which application is incorporated herein by reference for any and all purposes.

### TECHNICAL FIELD

The present disclosure relates to the field of point-of-care diagnostic devices.

### BACKGROUND

COVID-19 has to date killed many millions of people and has disproportionally affected individuals living in resource-limited settings, disadvantaged communities, and low-income countries. High-frequency testing represents a known tool for outbreak and viral spread prevention that can only be realized with accessible, low-cost, and rapid tests. Such a technology could have a major public health impact broadly, and in low-income countries in particular. However, the most widely used tests today, such as RT-PCR, are slow, relatively expensive, and require highly skilled workers and available lab space.

Existing COVID-19 diagnostic methods, however, require a long time and are relatively expensive, such as RT-PCR, which requires specialized labor and cannot be used without costly reagents and equipment. Accordingly, there is a long-felt need for improved viral diagnostic systems and methods.

### SUMMARY

Here, we report, inter alia, COvid-19 Low-cost Optodiagnostic for Rapid testing (COLOR), a SARS-COV-2 biosensor that uses highly accessible and inexpensive materials. The test can be easily assembled for 15¢ using cotton swabs modified with ACE2 to recognize the SARS-COV-2 spike protein (SP) and gold nanoparticles stabilized with cysteamine functionalized with human angiotensin-converting enzyme 2 (ACE2) (AuNP-cys-ACE2). COLOR enables COVID-19 diagnosis through a simple, minute-timescale color shift that can be detected by the naked eye and quantitative results may be generated using a smartphone camera and a free app. The technology displayed excellent accuracy (90%), sensitivity (96%), and selectivity (84%) in a panel of 100 clinical patient samples and provides a low-cost solution for COVID-19 testing.

As an exemplary embodiment of the disclosed technology, provided here is a SARS-COV-2 biosensor based on modified cotton swabs with ACE2 to recognize the SARS-COV-2 SP and gold nanoparticles stabilized with cysteamine functionalized with ACE2 (AuNP-cys-ACE2) to reveal the presence of the virus colorimetrically. The colorimetric device presents high accuracy (90%), sensitivity (96%), and selectivity (84%) for the detection of SARS-COV-2 in clinical samples. A qualitative colorimetric result can be quickly obtained by the naked eye, and quantitative results were obtained using a smartphone camera as a detector coupled to a colorimetric app to obtain the color intensity values (Red, Green, Blue (RGB)).

The use of colorimetric devices for detection has been boosted in the clinical field, mainly because those biosensors are easy to prepare, fast, portable, and count on metallic nanoparticles that have well-established optical properties. The field of research in colorimetric sensors has grown exponentially in the last few years as they present a fast and accurate response of the analyte of interest, through the color change, providing the individual with an easy-to-interpret colorimetric response. Furthermore, the device can be used by any individual without the need for training or specialized labor to interpret the results, being able to be employed in remote locations, points of care and domestic use.

In one aspect, the present disclosure provides a detection kit, comprising: a substrate having disposed thereon a first protein that selectively binds to a binding domain of a biomarker or antigen; and a supply of nanoparticles having disposed thereon a second protein that selectively binds to the binding domain of the biomarker or antigen.

Also provided are methods, comprising: the use of a kit according to the present disclosure (e.g., any one of Aspects 1-19) to detect the presence or absence of a virus in a subject's sample.

Further provided are methods, comprising: contacting a sample with a substrate having disposed thereon a first protein that selectively binds to a binding domain of a biomarker or antigen; contacting the substrate to a fluid comprising a supply of nanoparticles having disposed thereon a second protein that selectively binds to the binding domain of the biomarker or antigen; and monitoring a color of the fluid.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1a-1f—design, manufacturing, and characterization of COLOR. (a) Schematic representation of the synthesis steps needed to generate AuNPs, including their functionalization with cysteamine (cys) and ACE2. Briefly, cys was added in the presence of chloroauric acid for 20 min at room temperature and protected from light. Sodium borohydride was added to the solution at room temperature in a light-protected flask. Following, the AuNP-cys were exposed to a mixture of ACE2 and EDAC:NHS for functionalization of the enzyme on AuNP-cys surface. (b) Steps and real photos showing the colorimetric detection of SARS-COV-2 in an aqueous medium using the synthesized AuNP-cys-ACE2. Proof-of-principle of the recognition of SARS-COV-2 spike protein and the induced aggregation of the gold nanoparticles changing the solution color of wine for purple. (c) Schematic representation of the colorimetric steps needed for COVID-19 diagnosis using COLOR. In the presence of SARS-COV-2, a color shift occurs, and the cotton swabs change from white to purple color after 3 minutes. (d) Morphological characterization of the AuNP-cys solution, which presented a spherical shape and a high dispersion. (e) SEM image of AuNP-cys-ACE2 aggregation in the presence of SARS-CoV-2 SP. (f) Size histogram distribution obtained for AuNP-cys with a mean diameter size of 7.00±0.29 nm with a correlation coefficient (R²) of 0.94 was obtained.

FIG. 2a-2c—optimization studies of COLOR. (a) Study assessing the incubation period needed for functionalization of ACE2 onto the surface of the cotton swabs upon activation of the aldehyde groups with potassium periodate. The optimal incubation period to ensure ACE2 functionalization was determined to be 7 hours (b) Kinetic study of the interaction between the cotton swab-immobilized ACE2 and SP, which revealed an optimal incubation time of 1 min. (c) Study of incubation time between the cotton swab biosensor and AuNP-cys-ACE2, yielding an optimal time of 3 min for best colorimetric results. All results were optimized by evaluating the sensitivity parameters obtained from analytical curves using 1×10⁻¹¹ g mL⁻¹ to 1×10⁻⁸ g mL⁻¹ of SP in for 3 different biosensors.

FIG. 3a-3f—analytical parameters of COLOR obtained using a free app and a smartphone. (a) Image of the acquisition process of the color patterns from colorimetric cotton swabs biosensor using RGB app on the smartphone. (b) Analytical curve built using SP at concentrations ranging from 1×10⁻¹² g mL⁻¹ to 1×10⁻⁶ g mL⁻¹, with a linear behavior in the concentration ranging from 1×10⁻¹² g mL⁻¹ to 1×10⁻⁸ g mL⁻¹ SP. (c) Analytical curve correlating the color intensity (red pattern) with the Ct values (ranging from 25.3 Ct to 34.3 Ct) obtained by RT-PCR of positive SARS-COV-2 samples. (d) Selectivity studies using other coronaviruses and non-coronavirus strains; H₁N₁-A/California/2009 (10⁵ PFU mL⁻¹); Influenza-B/Colorado (10⁵ PFU mL⁻¹); Herpes simplex virus-2 (10⁸ PFU mL⁻¹) and MHV-murine hepatitis virus (10⁸ PFU mL⁻¹). Each virus was incubated in a final volume of 200 μL for 1 min at room temperature. (e) Reproducibility studies were carried out using 10 modified cotton swabs incubated for 1 min in the presence of 1×10⁻⁹ g mL⁻¹ SP, resulting in a relative standard derivation (RSD) of 5.70%. (f) Stability study of COLOR. The test was stored at 4° C. in PBS medium (pH=7.4) over 7 days with the best results obtained until 3 days. Sensitivity values were obtained through analytical curves generated at concentrations ranging between 1×10⁻¹² g mL⁻¹ to 1×10⁻⁹ mL⁻¹ SP. All experiments were carried out in triplicate (n=3 biosensors).

FIG. 4—UV-Vis spectrum of the synthesized AuNP-cys. The AuNP-cys suspension presented a maximum absorbance at 546 nm, which is characteristic of the plasmonic effect of gold nanoparticles. Therefore, this wavelength was used to study the long-term stability of AuNP-cys (FIG. 5).

FIG. 5—Stability study of the AuNP-cys solution. The stability study was evaluated by the mean values of the absorbance band at 546 nm extracted from UV-Vis spectra recorded over 5 days. The solution was stored at 4° C. in light-protected environment. Note that the AuNP-cys presented an adequate stability along the period evaluated.

FIG. 6—FTIR characterization of the activation process of the cotton swab. FTIR was used to evaluate the effectiveness of the activation protocol of the cotton surface [cotton swab was immersed in a 2.0 mmol L⁻¹ potassium periodate (KIO₄) solution containing 1.0 mL sulfuric acid overnight at room temperature] to generate aldehyde functional groups to enable the subsequent covalent immobilization of ACE2 on the surface of the cotton swab through the formation of an amide bond between the activated cotton and the enzyme using EDAC:NHS. Note that the FTIR spectrum of the cotton after the activation process with potassium periodate (red line) shows a clear an enhanced signal of the band at 1654 cm⁻¹, characteristic of the carbonyl group, compared to the spectrum obtained for the non-activated cotton swab (black line), confirming the successful generation of aldehyde functional groups.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

The COVID-19 pandemic has exacerbated society's tremendous health equity gap. Disadvantaged populations have been disproportionally affected by COVID-19, lacking access to affordable testing, a known effective tool for preventing viral spread, hospitalizations, and deaths. Here, we describe COvid-19 Low-cost Optodiagnostic for Rapid testing (COLOR), a colorimetric biosensor fabricated on cotton swabs using gold nanoparticles modified with human angiotensin-converting enzyme 2 (ACE2), which costs 15¢ to produce and detects SARS-COV-2 within 5 minutes. COLOR detected very low viral particle loads (e.g., limit of detection: 0.154 pg mL⁻¹ of SARS-COV-2 spike protein) and its color intensity correlated with the cycle threshold (Ct) values obtained using RT-PCR. The performance of COLOR was assessed using 100 nasopharyngeal/oropharyngeal (NP/OP) clinical samples, yielding sensitivity, specificity, and accuracy values of 96%, 84%, and 90%, respectively. In summary, each COLOR test can be manufactured for 15¢ and presents rapid minute-timescale detection of SARS-COV-2, thus providing a solution to enable high-frequency testing, particularly in low-resource communities.

### Exemplary Disclosure

In one embodiment used to illustrate the disclosed technology, we demonstrate COLOR with accessible cotton swabs as a convenient platform for at-home use. The colorimetric biosensor leverages ACE2 immobilized directly onto cotton to ensure selective and rapid recognition of SARS-COV-2 SP. The AuNP modified with ACE2 enables a visual readout of the results through the formation of a molecular sandwich with SARS-COV-2, which falls in between the cotton swab-ACE2 and the AuNP-ACE2.

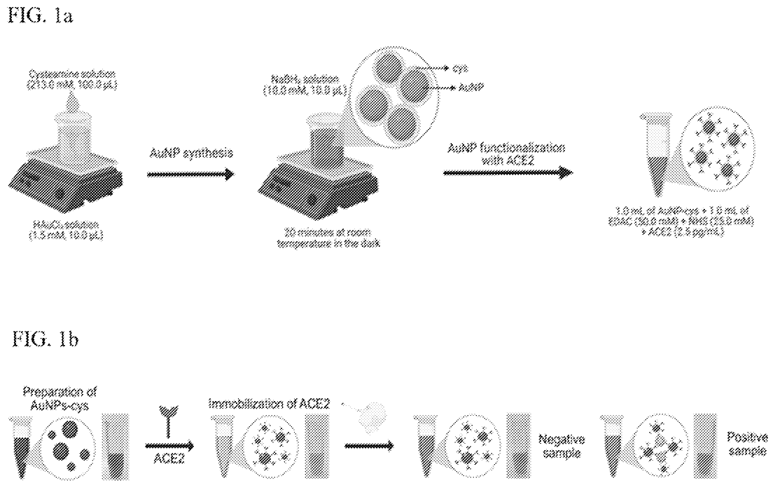

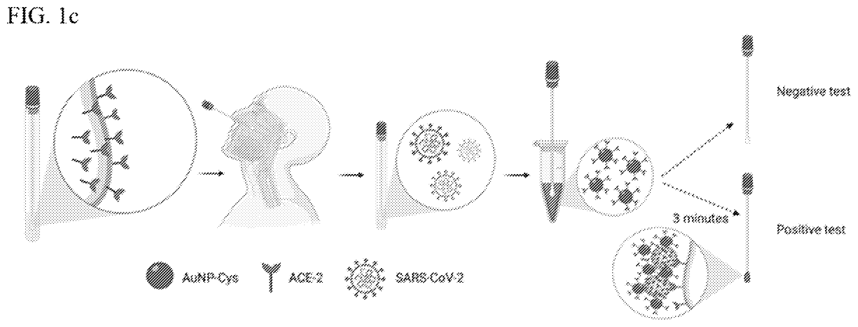

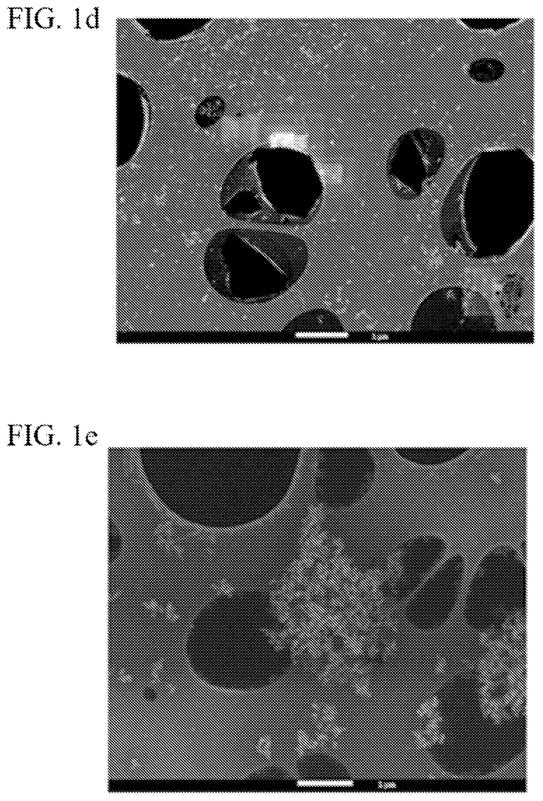

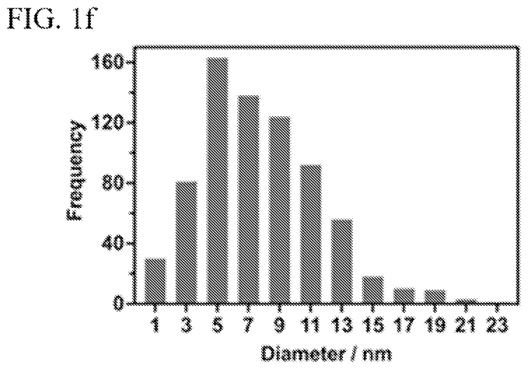

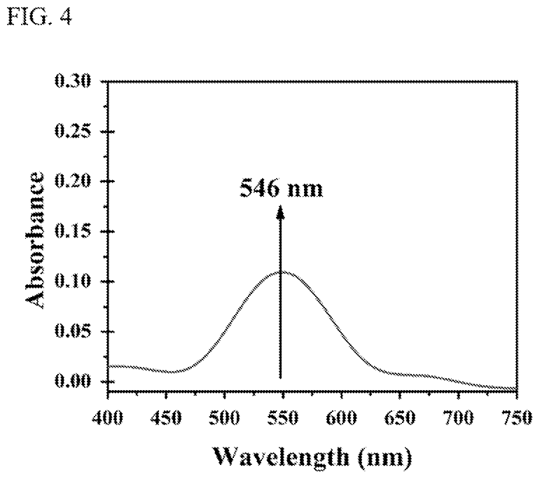

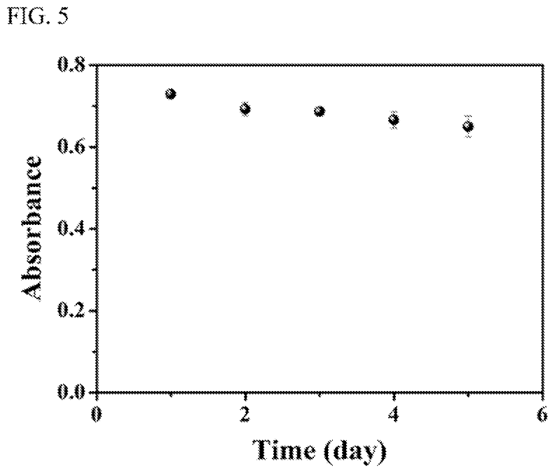

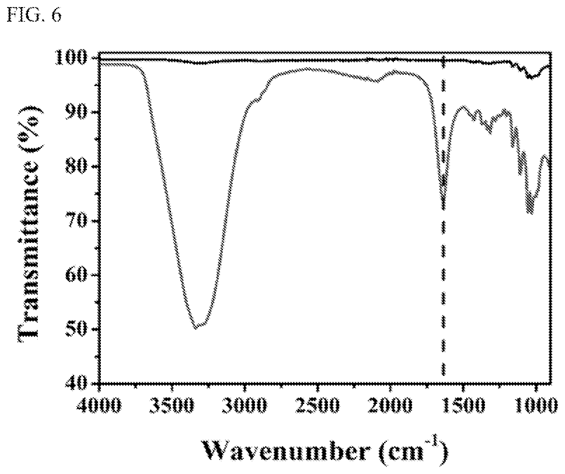

To manufacture COLOR, we first synthesized the AuNPs using cysteamine (cys) as a stabilizing agent (FIG. 1a). The UV-Vis spectrum obtained for AuNP-cys presented a maximum absorbance at 546 nm (FIG. 4), which is characteristic of the plasmonic effect of gold nanoparticles. Cys stabilizes AuNP by the covalent binding of thiol (—SH) groups onto the surface of the AuNPs through an Au—S bond. The termination of the amino-functional group of cys provides an electrostatic repulsion due to the protonation state (NH₃⁺), generating free positive charges that confer adequate stability to AuNPs when stored at 4° C. for up to 5 days (FIG. 5).

Next, the AuNP-cys were modified with ACE2 using N-(3-dimethylaminopropyl)-N-ethylcarbodiimide hydrochloride (EDAC) and N-Hydroxysuccinimide (NHS) to activate the carboxylic functional groups to then covalently bind to amino groups present in the AuNP-cys, generating an amide. This reaction generates a wine-light color solution that maintains its color when exposed to a negative COVID-19 sample (i.e., without SP) in VTM medium (FIG. 1c), demonstrating the stability and selectivity of the modified AuNPs. On the contrary, when exposed to SARS-CoV-2, the wine-colored solution shifts color to purple, resulting from the aggregation of AuNP-cys-ACE2 when in contact with SARS-COV-2 SP (FIG. 1c).

Collectively, the cotton swab modification process includes three steps: 1) overnight activation of the cotton surface with a potassium periodate solution containing 1 mL of sulfuric acid at room temperature to allow the formation of aldehyde groups on the cotton's surface through the conversion of poly-hydroxyl groups; 2) cotton functionalization with ACE2 using EDAC:NHS; and 3) blockage of non-specific sites and ensuring ACE2 stabilization by using bovine serum albumin (BSA) (FIG. 1d). The subsequent colorimetric detection involves immersion of the cotton swabs into standard SP solutions or clinical SARS-COV-2 samples. The SP in these samples bound to the surface of the cotton swabs due to its binding affinity toward ACE2. Subsequently, the cotton swab containing bound SARS-COV-2 SP was soaked into a AuNP-cys-ACE2 solution for 3 minutes. This step induced aggregation of the AuNP-cys-ACE2 onto the swab surface (positive COVID-19 test), and the wine-colored solution turned purple due to the formation of a molecular sandwich consisting of cotton-ACE2/SARS-COV-2 (or SP alone)/AuNP-ACE2. These results highlight the utility of AuNP-cys-ACE2 as a functional nanomaterial for diagnosing COVID-19.

We then performed thorough characterization studies of COLOR. Morphological characterization of AuNP-cys-ACE2 dispersion before (FIG. 1e) and after (FIG. 1f) incubation with SARS-COV-2 samples was conducted using scanning electron microscopy (SEM). The spherical AuNP-cys-ACE2 dispersion had an averaged size distribution with a diameter of 7.00±0.29 nm (FIG. 1g) that rapidly aggregated when exposed to SARS-COV-2 due to selective binding with ACE2. Binding to ACE2 yielded the formation of nanostructure aggregates with a mean diameter >200 nm, confirming the hypothesis that such an interaction would lead to a color shift (from wine-to-purple), observable by the naked eye.

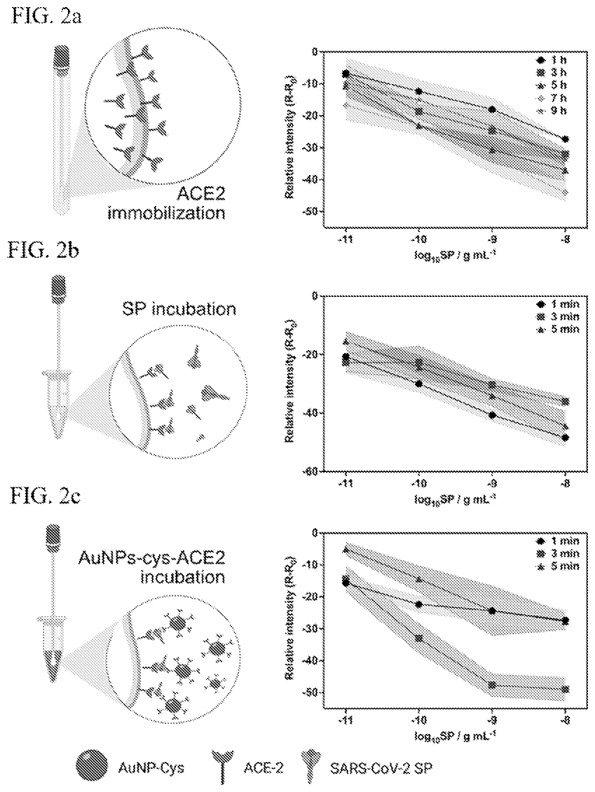

Once the proof-of-principle study was completed, we performed further optimization studies to obtain the best analytical signal (greater purple color intensity on cotton swabs), including optimization tests to enhance ACE2 immobilization onto the cotton swabs, assessment of the optimal time of incubation of the biosensors in the SP solution, and to enable the binding of the AuNP-cys-ACE2 solution to the cotton-SP complex providing the final color readout. The optimization process centered around obtaining the optimal conditions needed to yield the highest analytical sensitivity (angular coefficient) through calibration curves at concentrations ranging from 1×10⁻¹¹ g mL⁻¹ to 1×10⁻⁸ g mL⁻¹ of SP (FIG. 2a-c). ACE2 immobilization on the surface of the active cotton swab surface involved covalent anchoring of ACE2 through the formation of an amide bond, since the activation process of the cotton swabs with potassium periodate generated aldehyde functional groups confirmed by an increased band signal at 1654 cm⁻¹ using Fourier-transform infrared spectroscopy (FTIR) (FIG. 5). The amine terminal group of ACE2 bind to aldehyde functional groups present on the activated cotton surface leading to the formation of amide bonds, which enable a stable anchoring strategy for clinical analyses. Collectively, the experiments revealed that the optimal conditions needed for ACE anchoring, and consequently, analytical sensitivity (FIG. 2a), involved incubation of the cotton swab in an ACE2 solution for 7 hours.

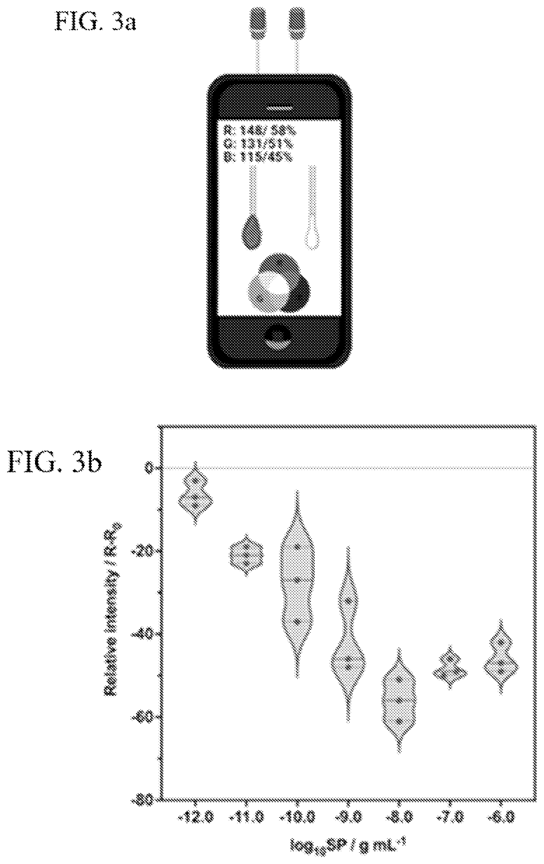

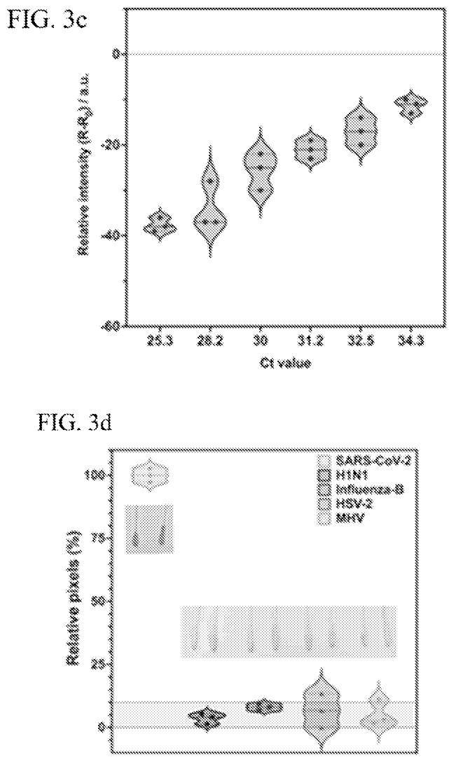

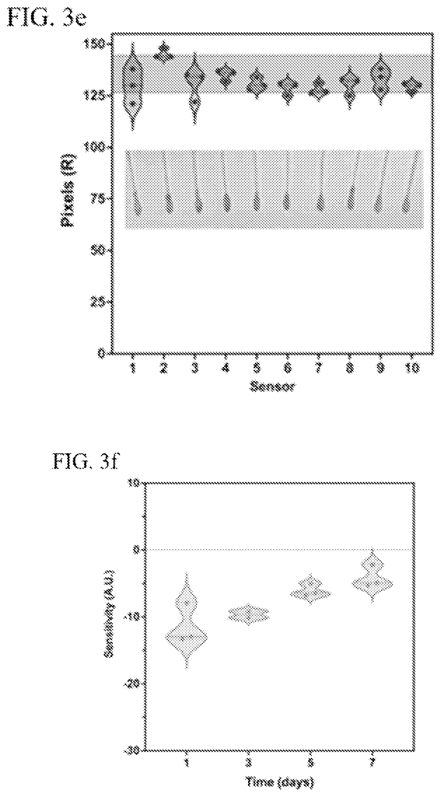

We next assessed the kinetics of the interaction between SP and the ACE2-modified cotton swab for an incubation period ranging from 1 to 5 min (FIG. 2b). We selected 1 min as the optimal condition as it yielded excellent sensitivity and analytical frequency (FIG. 2b). Finally, the cotton swabs exposed to SP solutions were incubated with AuNP-cys-ACE2, leading to a color change (FIG. 2c). The color readout is based on the selective binding of AuNP-cys-ACE2 to the surface of the cotton swab/ACE2/SP complex, leading to aggregation of the modified AuNPs on the functionalized cotton surface, which yields a purple color (FIG. 1c). This optimization process was carried out using an incubation period ranging from 1 to 5 min and an optimal sensitivity was obtained after 3 min (FIG. 2c). All colorimetric test images were captured using a smartphone camera (Samsung Galaxy J8 with Android system and camera 10.0.01.77), and the color patterns (RGB) were acquired by the open-access Color Detector app (FIG. 3a).

### Analytical Characterization Studies of COLOR

Once all experimental conditions were optimized (FIGS. 2a-c), we built an analytical curve by exposing the cotton swabs to solutions containing increasing concentrations of SP (10⁻¹² to 10⁻⁶ g mL⁻¹ SP) in VTM medium (FIG. 3b). Results were quantified using a smartphone camera by analyzing the color patterns (RGB) of the photos taken of the cotton swabs. The direct use of the red pattern intensity demonstrated the best correlation with the color obtained by the increase in SP concentration, i.e., the red pattern values decreased with increased SP concentration due to the red-shift triggered by aggregation of functionalized AuNPs. Thus, the red intensity signals (n=3 measurements) obtained at each concentration of SARS-COV-2 SP were plotted as a logarithmic function of the SP concentration, which ranged from 1×10⁻¹² g mL⁻¹ to 1×10⁶ g mL⁻¹. A linear behavior was observed in the range of 1×10⁻¹² g mL⁻¹ to 1×10⁻⁸ g mL⁻¹ of SP resulting in a sensitivity value of—12.14±0.06 pixels g⁻¹ mL and a linear regression (R²) of 0.99.

The limit of detection (LOD) and limit of quantification (LOQ) were obtained according to the Miller and Miller equation (Eq. 1), where the term (y₀-y_{B}) is the value of the intercept of the analytical curve after subtraction of the value of the blank solution (i.e., VTM medium), and S_{y/x} corresponds to the slope error. The LOQ was calculated by multiplying the LOD by a factor of 3.33, as previously described. The LOD and LOQ values of the modified cotton swabs were 0.154 pg mL⁻¹ and 0.513 pg mL⁻¹, respectively, underscoring the excellent performance and ultra-sensitive COVID-19 detection capabilities of COLOR. A side-by-side comparison of COLOR's performance with respect to other methods for SARS-COV-2 detection is presented in Table 1. Overall, COLOR displays an extremely low LOD, provides fast testing time (5 min total; 1 min of incubation with the clinical sample, 30 s first wash with PBS, 3 min incubation with the AuNP-cys-ACE2 solution, and an extra 30 s for the second and final wash with PBS), and is inexpensive to produce with a total cost of 15¢ (8¢ per plastic vial, 1 ¢ for recombinant ACE2, 1¢ for the cotton swab, and 5¢ for all the other reagents used).

To demonstrate the translational applications of COLOR, we tested its performance using 100 clinical samples containing different viral loads, each of which correlated with a specific cycle threshold (Ct) value as determined by RT-PCR. Ct values of the samples used ranged from 25.3 to 34.3 Ct (FIG. 3c). Of note, the colorimetric cotton swabs were able to discriminate high from very low viral loads and its results correlated with the Ct values obtained by RT-PCR.

LOD=(y₀−y_{B})+3_{sy/x}(Eq.1)

To verify the selectivity of COLOR toward SARS-COV-2, we performed cross-reactivity studies against other viruses. We tested four other viral strains: H₁N₁ (A/California/2009), Influenza-B/Colorado, Herpes simplex virus-2, and MHV-murine hepatitis virus. The results revealed no cross-reactivity against any of the viruses tested since all experiments yielded a response lower than 10% of the response obtained by the positive control sample [Red pixel (R-R₀), where R and R₀ denote the red color intensity for the sample and the analytical blank, respectively] (FIG. 3d).

Reproducibility assays were conducted to ensure different COLOR tests performed similarly. The colorimetric device was exposed to 1×10⁻⁹ mL⁻¹ of SP resuspended in a 0.1 mol L⁻¹ PBS solution (pH=7.4). A relative standard deviation (RSD) of 5.70% in the analytical signal (red color pattern) was obtained using ten different biosensors (n=10), indicating excellent reproducibility and suitability for high-frequency testing (FIG. 3e). Next, we evaluated the stability of COLOR when stored at 4° C. in PBS medium over 7 days. Analytical curves were generated in 0.1 mol L⁻¹ PBS (pH=7.4) with concentrations of SP ranging from 1×10⁻¹² g mL⁻¹ to 1×10⁻⁹ mL⁻¹ (FIG. 3f). COLOR remained functional after 7 days of storage (FIG. 3f). However, the mean sensitivity of the device decreased 50% when compared to the initial performance when it is used right after production (FIG. 3f).

Detection of SARS-CoV-2 in Clinical Samples with COLOR

To evaluate the diagnostic efficacy of the colorimetric cotton swabs, we tested their performance using 100 NP/OP (Table 2 and Table 3) clinical samples obtained from inpatients from the Hospital of the University of Pennsylvania (HUP). All samples were heat inactivated prior to testing and were confirmed as COVID-19 positive or COVID-19 negative by RT-PCR. Table 2 shows that out of the 100 NP/OP samples tested (50 COVID-19-positive and 50 COVID-19-negative samples), the device accurately detected 90 (90.0% accuracy).

### CONCLUSIONS

In this study, we describe COLOR, a low-cost, rapid, and easy-to-assemble COVID-19 test that uses highly accessible materials and provides a visual test readout that can be used to accurately diagnose SARS-COV-2 infections using a smartphone. Each test costs 15¢ to produce, generates a result within 5 min, is highly sensitive (e.g., LOD of 154 fg mL⁻¹ for SP), and demonstrated 90% accuracy in a study using 100 clinical samples. Collectively, COLOR represents an excellent solution to enable high-frequency testing, particularly in resource-limited populations.

### Materials and Methods

### Materials

All reagents used in the experiments presented analytical grade. The deionized water (resistivity ≥18 MΩ cm at 25° C.) was obtained from a Milli-Q Advantage-0.10 purification system (Millipore). Potassium periodate (KIO₄) was obtained by Beantown Chemical. Human angiotensin-converting enzyme 2 (ACE2) Fc Chimera was obtained from GenScript. Spike protein was kindly donated by Scott Hensley from the University of Pennsylvania. N-(3-dimethylaminopropyl)-N-ethylcarbodiimide hydrochloride (EDAC) and N-Hydroxysuccinimide (NHS) with a degree of purity ≥98%, gold (III) chloride trihydrate (HAuC14.3H2O) (99.99%), sodium borohydride (NaBH₄) with ≥98% purity, cysteamine hydrochloride (Cys) with 98% purity, phosphate buffer saline powder, pH=7.4 and glutaraldehyde (25%, v/v) were purchased from Sigma-Aldrich. Cotton swabs from the brand Q-tips were purchased in a drug store (Philadelphia/USA).

### Cotton Swabs Activation

The cotton surface's activation was carried out according to the following protocol. First, the cotton swabs were immersed in a 2.0 mmol L⁻¹ potassium periodate (KIO₄) solution containing 1.0 mL sulfuric acid overnight at room temperature. Then, the cotton swabs were washed with PBS solution (0.1 mol L⁻¹, pH=7.4) to remove the excess of oxidizing agent present on their surface, and thereafter, they were kept in PBS buffer prior to functionalization with ACE2. This procedure resulted in the formation of an aldehyde-substituted cotton surface by the conversion of poly-hydroxyl groups, which allowed subsequent anchoring of ACE2. The aldehyde groups present on the activated cotton swabs were confirmed by Fourier-transform infrared spectroscopy (FTIR).

Synthesis of the Cysteamine Stabilized Gold Nanoparticles and ACE2 Immobilization Studies

The gold nanoparticles were synthesized according to previous approaches, where the AuNPs were stabilized with cys (AuNP-cys) (FIG. 1a). Briefly, 100 μL of cysteamine (213.0 mmol L⁻¹) was added to 1.5 mmol L⁻¹ HAuCl₄ solution to a final volume of 10.0 mL. The solution was vigorously stirred at 1,200 rpm for 20 minutes at room temperature in a light-protected environment. Subsequently, 10.0 μL of NaBH₄ (10.0 mmol L⁻¹) were added to the solution and kept under stirring conditions for an extra 20 minutes in a light-protected flask and at room temperature, until the color of the solution changed from yellow to wine. Finally, the solution was stored at 4° C. in a refrigerator for up to 7 days. Next, 1.0 mL of AuNP-cys solution was modified with ACE2 (2.5 pg mL-1) for 30 minutes at room temperature in 1.0 mL of a solution containing EDAC (50.0 mmol L-1) and NHS (25.0 mmol L-1 in a final volume of 2.0 mL. This protocol enabled the formation of an amide bond between the AuNPs-cys surface and ACE2.

### Gold Nanoparticle Characterization Studies

Morphological characterization of gold nanoparticles stabilized with cysteamine (AuNP-cys) was performed using scanning electron microscopy (SEM). All images were acquired using a JEOL 7500F HRSEM microscope from the Singh Center for Nanotechnology (University of Pennsylvania). The Spectrophotometer measurements were carried out using a Perkin Elmer Multimode Plate Reader spectrophotometer (model En Vision). The absorbance of AuNP-cys was measured at 546 nm at room temperature. The effectiveness of cotton swab activation was evaluated by FTIR spectra obtained using a Perkin Elmer Spectrum 2 equipped with a Diamond UATR 2 detector with a scan range from 4,500 to 500 cm⁻¹ and a total of 32 scans by each measure.

ACE2 Immobilization onto the Surface of Cotton Swabs

First, cotton swabs were immersed in 200 μL of 1.25 pg mL⁻¹ ACE2 resuspended in 0.1 mol L⁻¹ PBS solution (pH=7.4). The incubation time was for 1, 3, 5, 7, and 9 hours at 4° C., and 7 hours was found to be the optimal condition for the anchoring of ACE2. Next, the swabs were rinsed to wash the unbounded ACE2. Subsequently, the swabs were resuspended for 30 minutes in a BSA 1% (m/v) solution in distilled water at 4° C. BSA was used to block the free aldehyde functional groups, i.e. which were not bound to ACE2, on the cotton surface.

### Colorimetric Assays

After the ACE2 and BSA modifications, the optimal incubation time of the cotton swabs in a SP solution was evaluated in order to obtain the best analytical signal. Analytical curves were generated at concentrations ranging between 10⁻¹¹ and 10⁻⁸ g mL⁻¹ of SP in 0.1 mol L⁻¹ PBS) using 1, 3, and 5 min of incubation at room temperature. The optimal incubation period was found to be 1 min, after which the cotton swabs were gently washed with a PBS solution over the 30s to remove the unbound excess of SP or SARS-COV-2 from clinical samples. To allow colorimetric detection using AuNP-cys-ACE2, we evaluated the incubation period of the cotton swabs into an AuNP-cys-ACE2 solution for 1, 3, and 5 min at room temperature. Exposure of SP or positive clinical samples to AuNP-cys-ACE2 solution over 3.0 min led to a visual response of the modified cotton swabs. Afterward, the cotton swabs were washed with a PBS solution for 30 s to remove the unbound excess of AuNP-cys-ACE2 solution to confirm the positive result. All colorimetric data were measured using the free app RGB (red, green, and blue) upon inspection of photos obtained using a smartphone (Samsung Galaxy J8 with Android system) camera 10.0.01.77.

### Reproducibility, Stability, and Cross-Reactivity Assays

Reproducibility studies were carried out using 10 different COLOR cotton swabs exposed to 1 ng mL⁻¹ of SP. Color intensity (red pattern) measurements were detected via the RGB app from photos of the cotton swabs taken after 1 min of the test. The stability of the modified cotton swabs was evaluated by the sensitivity value (slope) extracted from analytical curves at an interval range from 1×10⁻¹² to 1×10⁻⁶ g mL⁻¹ of SP for 1, 3, 5, and 7 days. Cross-reactivity assays were performed by exposing the modified cotton-swabs to 200 μL of five different viral samples: MHV—murine hepatitis virus at 10⁸ PFU mL⁻¹ (coronavirus), H₁N₁—A/California/2009, H₃N₂—A/Nicaragua, Influenza B—B/Colorado, HSV2-herpes simplex virus-2, all at 10⁵ PFU mL⁻¹. After the exposure period of 1 min between COLOR and each virus, the swabs were washed with PBS, exposed to AuNP-cys-ACE2 accordingly to the same protocol described for clinical sample analysis, and analyzed by the RGB app.

Analysis of Clinical Samples from Patients Using COLOR

One hundred nasopharyngeal/oropharyngeal (NP/OP) clinical samples (50 COVID-19 negative and 50 COVID-19 positive) resuspended in viral transport medium (VTM) were obtained from inpatients after heat-inactivation. All COVID-19 positive samples were confirmed by RT-PCR. We set a cut-off value of −5.0 for relative red pattern changes to determine as positive diagnostic, i.e., only changes lower than −5.0 in the red pixels were considered as positive for SARS-COV-2 infection.

### Aspects

The following Aspects are illustrative only and do not limit the scope of the present disclosure or the appended claims.

Aspect 1. A detection kit, comprising: a substrate having disposed thereon a first protein that selectively binds to a binding domain of a biomarker or an antigen; and a supply of nanoparticles having disposed thereon a second protein that selectively binds to the binding domain of the biomarker or antigen.

Aspect 2. The detection kit of claim 1, wherein the antigen is a virus, a bacteria, or a component thereof.

Aspect 3. The detection kit of claim 2, wherein the antigen comprises a viral spike protein.

Aspect 4. The detection kit of any one of claims 2-3, herein the virus is a coronavirus.

Aspect 5. The detection kit of Aspect 4, wherein the virus is SARS-COV-2.

Aspect 6. The detection kit of Aspect 1, wherein the first protein and the second protein are the same protein.

Aspect 7. The detection kit of any one of Aspects 1-6, wherein at least one of the first protein and the second protein is an enzyme.

Aspect 8. The detection kit of Aspect 7, wherein the enzyme is ACE-2.

Aspect 9. The detection kit of any one of Aspects 1-8, wherein the substrate comprises a pervious material.

Aspect 10. The detection kit of Aspect 9, wherein the substrate comprises cotton.

Aspect 11. The detection kit of any one of Aspects 1-10, wherein the substrate is characterized as being a swab.

Aspect 12. The detection kit of any one of Aspects 1-11, wherein the nanoparticles are functionalized so as to bind to the second protein.

Aspect 13. The detection kit of any one of Aspects 1-12, wherein the nanoparticles are metallic.

Aspect 14. The detection kit of Aspect 13, wherein the nanoparticles comprise gold. It should be understood that although gold nanoparticles were used to demonstrate the effectiveness of the disclosed technology, other nanoparticles can be used, including other metallic nanoparticles or other nanoparticles that exhibit suitable characteristics (e.g., surface plasmon resonance characteristics).

Aspect 15. The detection kit of any one of Aspects 1-12, wherein the nanoparticles comprise silica.

Aspect 16. The detection kit of any one of Aspects 1-12, wherein the nanoparticles are composite nanoparticles.

Aspect 17. The detection kit of any one of Aspects 1-16, further comprising a container in which the supply of nanoparticles is disposed, the container being disposed to receive the substrate.

Aspect 18. The detection kit of any one of Aspects 1-17, further comprising an imager configured to detect a color change or color intensity change associated with binding between the nanoparticles and the receptor binding domain of the viral spike protein.

Aspect 19. The detection kit of Aspect 18, wherein the imager comprises a mobile communications device.

Aspect 20. A method, comprising: the use of a kit according to any one of Aspects 1-19 to detect the presence or absence of a virus in a subject's sample.

Aspect 21. The method of Aspect 20, wherein the sample is a nasopharyngal or oropharyngeal sample.

Aspect 22. A method, comprising: contacting a sample with a substrate having disposed thereon a first protein that selectively binds to a binding domain of a biomarker or an antigen; contacting the substrate to a fluid comprising a supply of nanoparticles having disposed thereon a second protein that selectively binds to the binding domain of the biomarker or antigen; and monitoring a color of the fluid.

Aspect 23. The method of Aspect 22, further comprising correlating a change in the color of the fluid to the presence or the absence of the biomarker or antigen spike protein.

Aspect 24. The method of any one of Aspects 22-23, wherein the monitoring is effected in an automated fashion.

Aspect 25. The method of Aspect 24, wherein the monitoring is effected by a mobile computing device.

Aspect 26. The method of any one of Aspects 22-25, wherein the first protein and the second protein are the same protein.

Aspect 27. The method of any one of Aspects 22-26, wherein at least one of the first protein and the second protein is an enzyme.

Aspect 28. The method of Aspect 27, wherein the enzyme is ACE-2.

Aspect 29. The method of any one of Aspects 22-28, wherein the antigen is a virus, a bacteria, or a component thereof.

Aspect 30. The method of Aspect 29, wherein the antigen comprises a viral spike protein.

Aspect 31. The method of any one of Aspects 29-30, wherein the virus is a coronavirus.

Aspect 32. The method of Aspect 31, wherein the virus is SARS-COV-2.

Aspect 33. The method of any one of Aspects 22-32, wherein the substrate comprises a pervious material.

Aspect 34. The method of Aspect 33, wherein the substrate comprises cotton.

Aspect 35. The method of any one of Aspects 22-34, wherein the substrate is characterized as being a swab.

Aspect 36. The method of any one of Aspects 22-35, wherein the nanoparticles are functionalized so as to bind to the second protein.

Aspect 37. The method of any one of Aspects 22-30, wherein the nanoparticles are metallic.

Aspect 38. The method of Aspect 31, wherein the nanoparticles comprise gold.

Aspect 39. The method of any one of Aspects 22-36, wherein the nanoparticles comprise silica.

Aspect 40. The method of any one of Aspects 22-36, wherein the nanoparticles are composite nanoparticles.

1. A detection kit, comprising:
a substrate having disposed thereon a first protein that selectively binds to a binding domain of a biomarker or an antigen; and
a supply of nanoparticles having disposed thereon a second protein that selectively binds to the binding domain of the biomarker or antigen.

2. The detection kit of claim 1, wherein the antigen is a virus, a bacteria, or a component thereof.

3. The detection kit of claim 2, wherein the antigen comprises a viral spike protein.

4. The detection kit of claim 2, herein the virus is a coronavirus.

5. The detection kit of claim 4, wherein the virus is SARS-COV-2.

6. The detection kit of claim 1, wherein the first protein and the second protein are the same protein.

7. The detection kit of claim 1, wherein at least one of the first protein and the second protein is an enzyme.

8. The detection kit of claim 7, wherein the enzyme is ACE-2.

9. The detection kit of claim 1, wherein the substrate comprises a pervious material.

10. (canceled)

11. The detection kit of claim 1, wherein the substrate is characterized as being a swab.

12. The detection kit of claim 1, wherein the nanoparticles are functionalized so as to bind to the second protein.

13. The detection kit of claim 1, wherein the nanoparticles are metallic.

14. The detection kit of claim 13, wherein the nanoparticles comprise gold.

15. The detection kit of claim 1, wherein the nanoparticles comprise silica.

16. The detection kit of claim 1, wherein the nanoparticles are composite nanoparticles.

17. The detection kit of claim 1, further comprising a container in which the supply of nanoparticles is disposed, the container being disposed to receive the substrate.

18. The detection kit of claim 1, further comprising an imager configured to detect a color change or color intensity change associated with binding between the nanoparticles and the receptor binding domain of the viral spike protein.

19. The detection kit of claim 18, wherein the imager comprises a mobile communications device.

20. A method, comprising: the use of a kit according to claim 1 to detect the presence or absence of a virus in a subject's sample.

21. The method of claim 20, wherein the sample is a nasopharyngal or oropharyngeal sample.

22. A method, comprising:
contacting a sample with a substrate having disposed thereon a first protein that selectively binds to a binding domain of a biomarker or an antigen:
contacting the substrate to a fluid comprising a supply of nanoparticles having disposed thereon a second protein that selectively binds to the binding domain of the biomarker or antigen; and
monitoring a color of the fluid.

23. The method of claim 22, further comprising correlating a change in the color of the fluid to the presence or the absence of the biomarker or antigen spike protein.

24. The method of claim 22, wherein the monitoring is effected in an automated fashion.

25. The method of claim 24, wherein the monitoring is effected by a mobile computing device.

26. The method of claim 22, wherein the first protein and the second protein are the same protein.

27. The method of claim 22, wherein at least one of the first protein and the second protein is an enzyme.

28. The method of claim 27, wherein the enzyme is ACE-2.

29. The method of claim 22, wherein the antigen is a virus, a bacteria, or a component thereof.

30. The method of claim 29, wherein the antigen comprises a viral spike protein.

31. The method of claim 29, wherein the virus is a coronavirus.

32. The method of claim 31, wherein the virus is SARS-COV-2.

33. The method of claim 22, wherein the substrate comprises a pervious material.

34. (canceled)

35. The method of claim 22, wherein the substrate is characterized as being a swab.

36. The method of claim 22, wherein the nanoparticles are functionalized so as to bind to the second protein.

37. The method of claim 22, wherein the nanoparticles are metallic.

38. The method of claim 31, wherein the nanoparticles comprise gold.

39. The method of claim 22, wherein the nanoparticles comprise silica.

40. The method of claim 22, wherein the nanoparticles are composite nanoparticles.
